(19) United States
(12) Patent Application Publication
(10) Pub. No.: US 2026/0257010 A1
(43) Pub. Date: Sep. 3, 2026

(54) ADHESIVE PATCH

(71) Applicant: Unomedical A/S, Lejre (DK)

(72) Inventor: Erik Othel-Jacobsen, Snekkersten (DK)

(21) Appl. No.: 19/656,361

(22) Filed: Apr. 23, 2026

Related U.S. Application Data
Application No. 17/727,185 is a continuation of application No. PCT/EP2022/060649, filed on Apr. 22, 2022.
(62) Division of application No. 17/727,185, filed on Apr. 22, 2022, now abandoned.
(60) Provisional application No. 63/178,808, filed on Apr. 23, 2021.

(51) Int. Cl. A61M 5/142

(52) U.S. Cl. CPC A61M 5/14248

(57) ABSTRACT

An exemplary apparatus generally includes a medical device and an adhesive patch. The medical device is configured to be placed on a patient, and includes a base portion comprising a patient-facing side configured to face toward patient skin during use of the medical device. The adhesive patch is configured to adhere the base portion to patient skin. The adhesive patch includes a first section and a second section. The first section and the second section are separable from one another such that the first section is operable to be removed for replacement while the second section maintains the medical device in a particular position relative to the patient skin.

### CROSS-REFERENCE TO RELATED DISCLOSURES

The present disclosure is a divisional of U.S. Application No. 17/727,185 filed April 22, 2022, which was a continuation of International Application No. PCT/EP2022/060649 filed on April 22, 2022 and claims the benefit of U.S. Provisional Application No. 63/178,808 filed April 23, 2021, the contents of which being incorporated herein by reference in entirety.

### TECHNICAL FIELD

The present disclosure generally relates to adhesive patches, and more particularly but not exclusively relates to medical-grade adhesive patches configured for use with infusion devices.

### BACKGROUND

Infusion devices are often utilized to provide a therapeutic agent to a patient over an extended period of time, typically one to three days. Such infusion devices are commonly adhered to the skin via an adhesive patch. Many conventional adhesive patches suffer from one or more drawbacks or limitations. For example, many conventional adhesive patches will peel around the edges thereof, which may require the user to apply additional adhesive to prevent the infusion device from being dislodged from the application site. Additionally, many conventional adhesive patches cannot be replaced while maintaining the position of the infusion device, and require that the needle be withdrawn and reinserted, which may lead to additional discomfort for the patient. Moreover, many conventional adhesive patches obscure the infusion site, which inhibits the user from inspecting the condition of the infusion site. For these reasons among others, there remains a need for further improvements in this technological field.

### SUMMARY

An exemplary apparatus generally includes a medical device and an adhesive patch. The medical device is configured to be placed on a patient, and includes a base portion comprising a patient-facing side configured to face toward patient skin during use of the medical device. The adhesive patch is configured to adhere the base portion to patient skin. The adhesive patch includes a first section and a second section. The first section and the second section are separable from one another such that the first section is operable to be removed for replacement while the second section maintains the medical device in a particular position relative to the patient skin. Further embodiments, forms, features, and aspects of the present application shall become apparent from the description and figures provided herewith.

### BRIEF DESCRIPTION OF THE FIGURES

FIG. 1 is an exploded assembly view of an apparatus according to certain embodiments.

FIG. 2 is a cutaway view of an adhesive patch according to certain embodiments, which may be utilized in the apparatus illustrated in FIG. 1.

FIG. 3 is an exploded assembly view of an apparatus according to certain embodiments.

FIG. 4 is a cutaway view of an adhesive patch according to certain embodiments, which may be utilized in the apparatus illustrated in FIG. 3.

FIG. 5 is a perspective view of an adhesive patch according to certain embodiments.

FIG. 6 is a cutaway view of the adhesive patch illustrated in FIG. 5.

FIG. 7 is an exploded assembly view of an apparatus according to certain embodiments.

FIG. 8 is a cutaway view of an adhesive patch according to certain embodiments, which may be utilized in the apparatus illustrated in FIG. 7.

FIG. 9 is a schematic representation of a hook-loop fastener that may be utilized in certain embodiments.

FIG. 10 is a schematic representation of a hook-hook fastener that may be utilized in certain embodiments.

FIG. 11 is a schematic representation of a snap fastener that may be utilized in certain embodiments.

FIG. 12 is a schematic representation of a synthetic setae fastener that may be utilized in certain embodiments.

### DETAILED DESCRIPTION OF ILLUSTRATIVE EMBODIMENTS

Although the concepts of the present disclosure are susceptible to various modifications and alternative forms, specific embodiments have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to "one embodiment," "an embodiment," "an illustrative embodiment," etc., indicate that the embodiment described may include a particular feature, structure, or characteristic, but every embodiment may or may not necessarily include that particular feature, structure, or characteristic. Moreover, such phrases are not necessarily referring to the same embodiment. It should further be appreciated that although reference to a "preferred" component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described.

Additionally, it should be appreciated that items included in a list in the form of "at least one of A, B, and C" can mean (A); (B); (C); (A and B); (B and C); (A and C); or (A, B, and C). Similarly, items listed in the form of "at least one of A, B, or C" can mean (A); (B); (C); (A and B); (B and C); (A and C); or (A, B, and C). Items listed in the form of "A, B, and/or C" can also mean (A); (B); (C); (A and B); (B and C); (A and C); or (A, B, and C). Further, with respect to the claims, the use of words and phrases such as "a," "an," "at least one," and/or "at least one portion" should not be interpreted so as to be limiting to only one such element unless specifically stated to the contrary, and the use of phrases such as "at least a portion" and/or "a portion" should be interpreted as encompassing both embodiments including only a portion of such element and embodiments including the entirety of such element unless specifically stated to the contrary.

In the drawings, some structural or method features may be shown in certain specific arrangements and/or orderings. However, it should be appreciated that such specific arrangements and/or orderings may not necessarily be required. Rather, in some embodiments, such features may be arranged in a different manner and/or order than shown in the illustrative figures unless indicated to the contrary. Additionally, the inclusion of a structural or method feature in a particular figure is not meant to imply that such feature is required in all embodiments and, in some embodiments, may be omitted or may be combined with other features.

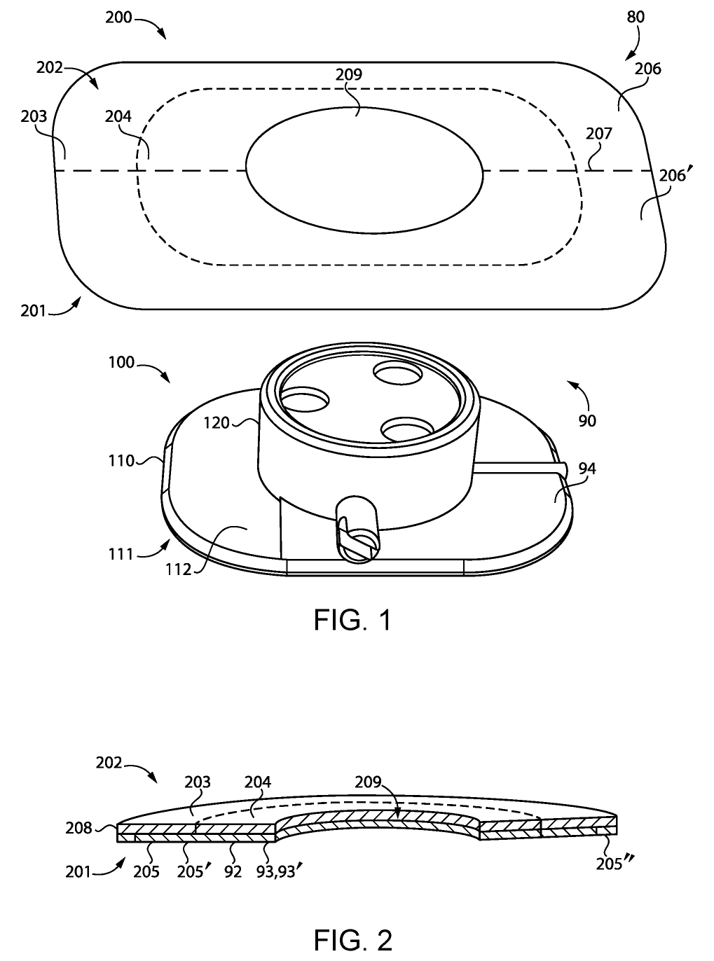

With reference to FIG. 1, illustrated therein is an apparatus 80 according to certain embodiments. The apparatus 80 generally includes a medical device 100 configured to be placed on a patient, an adhesive patch 200 configured to adhere the medical device 100 to the skin of the patient, and an attachment device 90 attaching the medical device 100 and the adhesive patch 200.

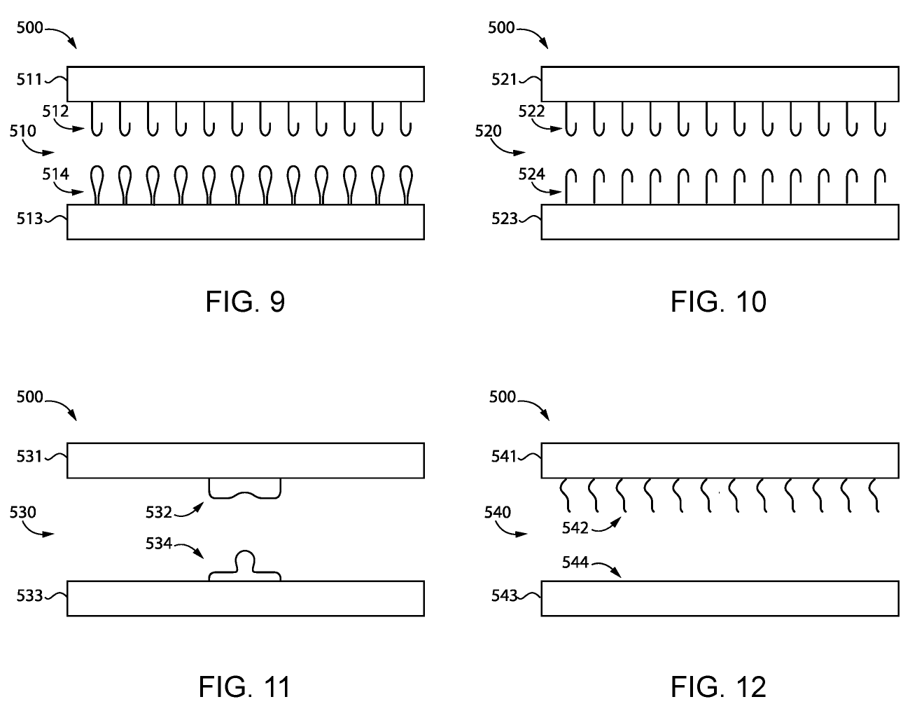

As described herein, the attachment device 90 generally includes a first attachment member 92 provided to the adhesive patch 200 and a second attachment member 94 that is provided to the medical device 100, and which is configured for releasable attachment with the first attachment member 92. In certain embodiments, the first attachment member 92 comprises an adhesive layer 93 comprising an attachment member adhesive composition 93'. In certain such forms, the attachment member adhesive composition 93' is different from an adhesive composition 205' by which a first region 203 of the adhesive patch 200 may be adhered to patient skin. In addition or as an alternative to a second adhesive composition 93' different from the first adhesive composition 205', the attachment device 90 may include a mechanical fastener. Further details regarding additional exemplary forms for the attachment device 90 are provided herein with reference to FIGS. 9-12.

The medical device 100 generally includes a base portion 110 and a housing 120 that houses the working components of the medical device 100. While the illustrated medical device 100 is provided in the form of an infusion device, it is also contemplated that the adhesive patches described herein may be utilized to secure other forms of medical device to patient skin.

The base portion 110 generally includes a patient-facing side 111 configured to face in a direction of patient skin during use of the device 100, and a second side 112 opposite the patient- facing side 111. In the illustrated form, the second side 112 comprises the second attachment member 94 of the attachment device 90. For example, the second side 112 may define the second attachment member 94, or the second attachment member 94 may be secured (e.g., adhered) to the second side 112.

The housing 120 is positioned on the second side 112 of the base portion 110 and houses the working components of the medical device 100. As noted above, the illustrated medical device 100 is an infusion device. As such, the housing 120 may house a reservoir of therapeutic agent and a needle operable to subcutaneously transmit the therapeutic agent to the patient. It is also contemplated that the housing 120 may house additional or alternative components, for example in embodiments in which the medical device 100 is provided in a form other than that of an infusion device.

With additional reference to FIG. 2, the adhesive patch 200 generally includes a first or patient-facing side 201 and an opposite second side 202. The adhesive patch 200 also includes a first region 203 configured for contacting patient skin and a second region 204 attached to the base portion 110 via the attachment device 90. More particularly, the second region 204 comprises the first attachment member 92 of the attachment device 90. In the illustrated form, the first region 203 and the second region 204 are provided on the patient-facing side 201, and the first region 203 at least partially surrounds the second region 204. As described herein, it is also contemplated that the first region 203 and the second region 204 may be positioned on opposite sides of the adhesive patch 200, and that the first region 203 may not necessarily surround the second region 204.

The first region 203 comprises a first region adhesive layer 205 comprising a first region adhesive composition 205' configured to adhere to patient skin. The second region 204 comprises the first attachment member 92 of the attachment device 90. In certain embodiments, the second region 204 may be more rigid than the first region 203. Such varying rigidity of the substrate 208 may facilitate a more even attachment of the second region 204 to the base portion 110 while enabling the first region 203 to flex according to patient movement.

As noted above, certain embodiments of the first attachment member 92 comprise an adhesive layer 93 comprising an adhesive composition 93'. In certain such embodiments, the first region adhesive composition 205' may have a greater adhesive strength than the first attachment member adhesive composition 93'. Such differences in adhesive properties may enable the first region adhesive layer 205 to securely adhere to skin while facilitating removal of the adhesive patch 200 from the medical device 100 during replacement of the patch 200. In certain embodiments, the outer periphery of the first adhesive layer 205 may comprise a second adhesive composition 205" having a greater adhesive strength than the first adhesive composition 205' to discourage the edges from peeling off of patient skin after application.

The illustrated adhesive patch 200 comprises a first section 206 and a second section 206', each of which comprises a corresponding and respective portion of the first region 203 and a corresponding and respective portion of the second region 204. The patch 200 may include one or more perforations 207 that facilitate separation of the first section 206 and the second section 206'. In the illustrated form, the perforations 207 divide the patch 200 into two substantially identical sections 206, 206'. It is also contemplated that the sections 206, 206' may not necessarily be identical, and that the perforations 207 may divide the patch 200 into three or more sections.

The patch 200 also includes a substrate 208 that defines an opening 209 operable to receive the housing 120. The patient-facing side 201 and the second side 202 are defined on opposite sides of the substrate 208. In certain embodiments, the adhesive patch 200 may further comprise a protective film that covers the adhesive layer 205 and/or the first attachment device 92 prior to use. In use of the apparatus 80, the medical device 100 may be placed on the patient skin in the appropriate position. Either before, during, or after insertion of the needle into the patient skin, the medical device 100 may be secured to the patient skin using the adhesive patch 200. In embodiments that include the protective film, the protective film may be removed to thereby expose the adhesive layer 205 and/or the first attachment device 92. The adhesive patch 200 may then be positioned such that the housing 120 extends through the opening 209 while the first region 203 contacts patient skin and the second region 204 contacts the second side 112 of the base portion 110. In such a position, the first adhesive layer 205 adheres the patch 200 to the skin while the attachment device 90 secures the adhesive patch 200 to the base portion 110. As a result, the position of the medical device 100 relative to patient skin is maintained by the adhesive patch.

In use, it may be the case that some performance characteristic of the adhesive patch 200 begins to degrade while the medical device 100 remains useful in its installed position. For example, the edges of the first adhesive layer 205 may have begun to peel from the patient skin. While such peeling may be discouraged by providing the adhesive layer 205 with a stronger adhesive composition 205" about its periphery as described above, it may nonetheless become desirable to replace the patch 200 while maintaining the position of the medical device 100. In such circumstances, the features of the illustrated patch 200 may aid in maintaining the position of the medical device 100 during replacement of the patch 200. For example, a user may be provided with a replacement adhesive patch 200 and may separate the replacement adhesive patch 200 into its first and second sections 206, 206', for example by tearing the patch 200 along its perforations 207. The user may then remove the first section 206 of the applied adhesive patch 200 for replacement while maintaining the second section 206' in its applied state such that the second section 206' maintains the medical device 100 in its installed position. After application of the first section 206 of the replacement adhesive patch 200, the user may remove the second section 206' of the original adhesive patch 200 for replacement while maintaining the first section 206 of the new adhesive patch 200 in its applied state to thereby maintain the medical device 100 in its installed position while replacing the second section 206'.

As should be evident from the foregoing, the illustrated adhesive patch 200 is configured to facilitate its own replacement while maintaining the medical device 100 in its installed position during such replacement. As a result, the needle of the infusion device 100 may remain embedded during replacement of the patch 200, which may reduce patient discomfort by obviating the need for reinserting the needle.

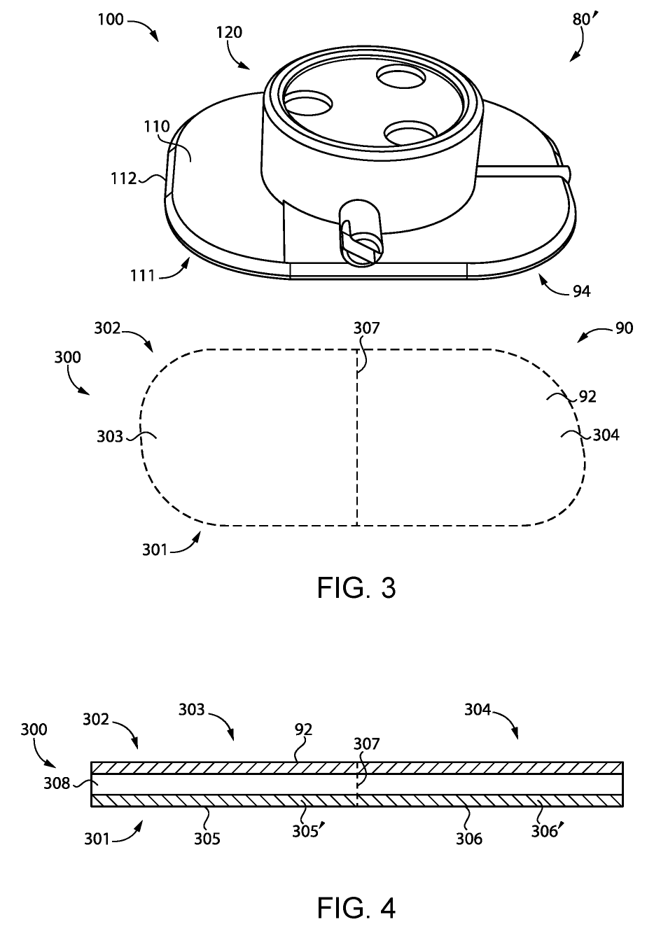

With additional reference to FIG. 3, illustrated therein is an apparatus 80' according to certain embodiments. Like the above-described apparatus 80, the apparatus 80' includes the medical device 100. The apparatus 80' also includes an adhesive patch 300 according to certain embodiments, and the attachment device 90 attaches the adhesive patch 300 to the medical device.

With additional reference to FIG. 4, the adhesive patch 300 generally includes a patient- facing first side 301 and an opposite second side 302, which are formed on opposite sides of a substrate 308. The patch 300 includes a first section 303 and a second section 304 and may further include a perforation 307 to facilitate separation of the first section 303 and the second section 304. In the illustrated form, the perforation 307 divides the patch 300 into two substantially identical sections 303, 304. It is also contemplated that the sections 303, 304 may not necessarily be identical, and that the perforations 207 may divide the patch 300 into three or more sections.

Formed on the first side 301 of the first section 303 is a first adhesive layer 305 comprising a first adhesive composition 305'. Formed on the first side 301 of the second section 304 is a second adhesive layer 306 comprising a second adhesive composition 306'. The second side 302 comprises the first attachment member 92. The first attachment member 92 is partially formed in the first section 303 and partially formed in the second section 304 such that each of the first section 303 and the second section 304 comprises a corresponding and respective portion of the first attachment member 92. In certain forms, the attachment device 90 may comprise an adhesive composition different from the first adhesive composition 305' and/or the second adhesive composition 306'. In addition, or as an alternative, the attachment device 90 may include a mechanical fastener. Further details regarding additional exemplary forms for the attachment device 90 are provided herein with reference to FIGS. 9-12.

In the illustrated form, the patient-facing side 111 of the base portion 110 comprises the second attachment member 94. As such, in the current embodiment, the attachment device 90 is operable to attach the patient-facing first side 111 of the base portion 110 with the second side 302 of the adhesive patch 300. In the illustrated form, substantially all of the adhesive patch 200 is positioned between the base portion 110 and the patient skin such that the base portion 110 covers substantially all of the adhesive patch 200. It is also contemplated that a portion (e.g., a tab) of one or both sections 303, 304 may project beyond the outer periphery of the base portion 110.

It should be appreciated that the adhesive patch 300 comprising plural sections 303, 304 may facilitate its own replacement in a manner analogous to that described above with reference to the adhesive patch 200 comprising plural sections 206, 206'. For example, a user may be provided with a replacement adhesive patch 300 and may separate the replacement adhesive patch 300 into its first and second sections 303, 304, for example by tearing the patch 300 along its perforation(s) 307. The user may then remove the first section 303 of the applied adhesive patch 300 for replacement while maintaining the second section 304 in its applied state such that the second section 304 maintains the medical device 100 in its installed position. After application of the first section 303 of the replacement adhesive patch 300, the user may remove the second section 304 of the original adhesive patch 300 for replacement while maintaining the first section 303 of the new adhesive patch 303 in its applied state to thereby maintain the medical device 100 in its installed position while replacing the second section 304.

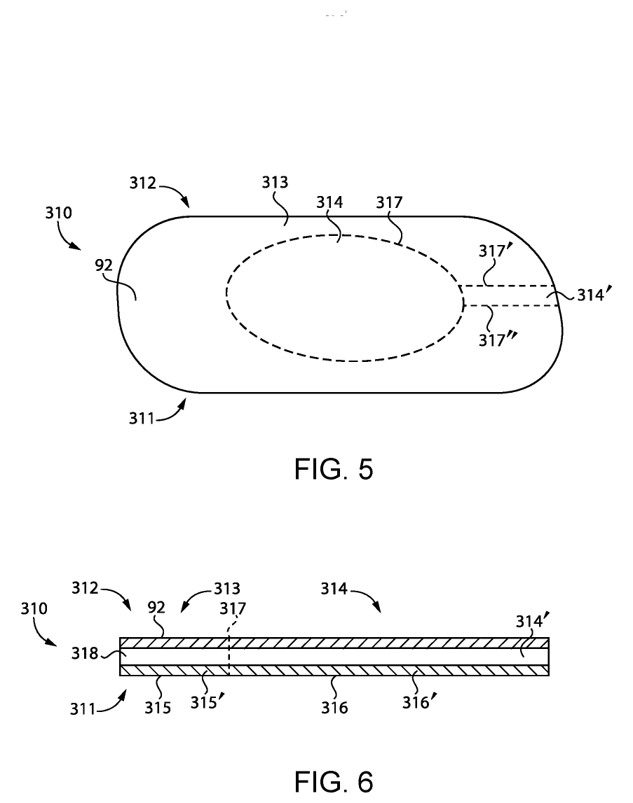

With additional reference to FIGS. 5 and 6, illustrated therein is an adhesive patch 310 according to certain embodiments. The adhesive patch 310 may, for example, be utilized in the apparatus 80' in place of the above-described adhesive patch 300. The patch 310 is somewhat similar to the patch 300, and similar reference characters are used to denote similar elements and features. For example, the patch 310 includes a substrate 318, first side 311, and opposite second side 312, a first section 313, a second section 314, and perforations 317 facilitating separation of the first section 313 and the second section 314. The first side 311 of the first section 313 includes a first adhesive layer 315 comprising a first adhesive composition 315', and the first side 311 of the second section 314 includes a second adhesive layer 316 comprising a second adhesive composition 316'. In the interest of conciseness, the following descriptions of the patch 310 focus primarily on features that are different from those described above with reference to the patch 300.

In the illustrated form, the first section 313 at least partially surrounds the second section 314, and a set of primary perforations 317 is defined at a boundary between the first section 313 and the second section 314. In certain embodiments, an additional perforation 317' extends from the primary perforations 317 toward the periphery of the patch 310 to facilitate removal of the first section 313. In certain embodiments, a further perforation 317" extends alongside the additional perforation 317' such that the second section 314 includes an extension 314' defined between the additional perforation 317' and the further perforation 317". In certain embodiments, the first adhesive composition 315' and the second adhesive composition 316' are different from one another. For example, the first adhesive composition 315' may have a stronger adhesive strength than the second adhesive composition 316'. Such an arrangement may cause the stronger adhesive composition 315' to discourage peeling about the edges of the patch 310 while the weaker adhesive composition 316' permits additional movement relative to the skin. In certain embodiments, the first adhesive composition 315' may be a non- silicone adhesive composition selected for durability. In certain embodiments, the second adhesive composition 316' may be a silicone-like adhesive that increases in strength over time.

In certain embodiments, the substrate 318 may exhibit different characteristics in the first section 313 as compared to the second section 314. For example, the substrate 318 in the first section 313 may be formed of a first material and/or have a first weave pattern, and the substrate in the second section 314 may be formed of a second material and/or have a second weave pattern. For example, the substrate 318 in the first section 313 may be formed of a more rigid material and/or have a tighter weave pattern to discourage peeling, while the substrate 318 in the second section 314 may be formed of a less rigid material and/or have a looser weave pattern to provide greater flexibility and/or breathability for the patient skin.

The second side 312 of the patch 310 includes the first attachment member 92, which is positioned at least partially in the first section 313. For example, the first attachment member 92 may extend about a periphery of the first section 313 to discourage peeling from the base portion 110. In certain embodiments, the attachment member 92 may be positioned partially in the second section 314, for example to enhance the attachment with the base portion 110. In other embodiments, the attachment member 92 may be omitted from the second section 314 to enhance breathability for the patient skin.

It should be appreciated that the adhesive patch 310 comprising plural sections 313, 314 may facilitate its own replacement in a manner analogous to that described above with reference to the adhesive patch 200 comprising plural sections 206, 206'. For example, a user may be provided with a replacement adhesive patch 310 and may separate the replacement adhesive patch 310 into its first and second sections 313, 314, for example by tearing the patch 310 along its perforation 317, 317', 317". The user may then remove the first section 313 of the applied adhesive patch 310 for replacement while maintaining the second section 314 in its applied state such that the second section 314 maintains the medical device 100 in its installed position.

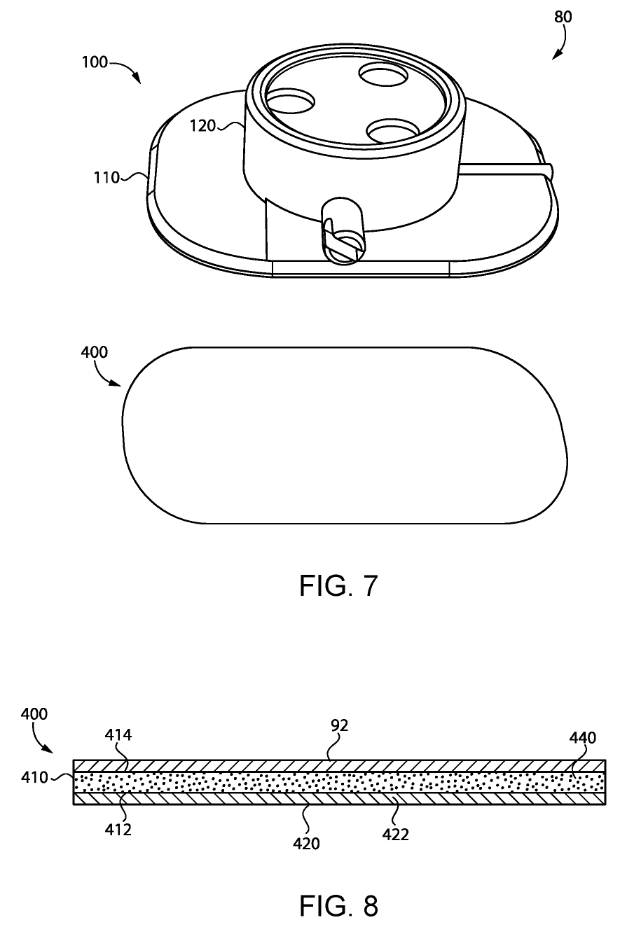

With additional reference to FIG. 7, illustrated therein is an apparatus 80" according to certain embodiments. Like the above-described apparatus 80, the apparatus 80" includes the medical device 100. The apparatus 80" also includes an adhesive patch 400 according to certain embodiments, and the attachment device 90 attaches the adhesive patch 400 to the medical device.

With additional reference to FIG. 8, the adhesive patch 400 generally includes a substrate 410 having a patient-facing first side 412 and an opposite second side 414. A first adhesive layer 420 is provided on the first side 412, and the first attachment member 92 is provided on the second side 414. The first adhesive layer 420 comprises a first adhesive composition 422. The first attachment member 92 comprises at least a portion of a mechanical fastener and/or a second adhesive composition different from the first adhesive composition 422. Further details regarding example embodiments of the attachment device 90 are provided below with reference to FIGS. 9-12.

The illustrated adhesive patch 400 further comprises a color-changing composition 440 configured to change colors when exposed to a particular fluid. In the illustrated form, the color- changing composition 440 is provided in the substrate 410. It is also contemplated that the color-changing composition 440 may be provided in the first adhesive layer 420 or elsewhere on and/or in the adhesive patch 400. While the adhesive patch 400 illustrated in FIGS. 7 and 8 is provided as a single-piece patch that lacks perforations, it should be appreciated that the color- changing composition 440 may be provided in multi-section patches, such as those described above with reference to FIGS. 1-6.

As noted - above, the color-changing composition 440 is configured to change colors when exposed to a particular fluid. In certain forms, the particular fluid is air, and the color-changing composition 440 is configured to change colors when exposed to air for a predetermined time period. In certain embodiments, the predetermined time period corresponds to an effective life of the medical device 100. For example, if the infusion device 100 holds a quantity of therapeutic agent sufficient to last for three days, the color-changing composition 440 may change colors when exposed to air for durations of three days or longer to thereby indicate to the patient that the infusion device 100 should be reloaded or replaced. As will be appreciated, the patch 400 may be kept in a vacuum-sealed package prior to use such that the three-day time period does not begin until the package is opened for application of the patch 400.

In certain embodiments, the particular fluid that causes the color-changing composition 440 to change colors is the therapeutic agent being dispersed by the infusion device 100. For example, if the infusion device 100 carries an insulin product, the color-changing composition 440 may change colors when exposed to the insulin product to thereby indicate to the user that a leak has occurred. As another example, if the infusion device 100 carries a chemotherapy product, the color-changing composition 440 may change colors when exposed to the chemotherapy product to thereby indicate to the user that a leak has occurred.

With additional reference to FIGS. 9-12, illustrated therein are various embodiments of mechanical fasteners 50. that may, for example, be utilized in connection with the attachment devices 90 described herein. More particularly, FIG. 9 illustrates a mechanical fastener 500 in the form of a hook-loop fastener 510, FIG. 10 illustrates a mechanical fastener 500 in the form of a hook-hook fastener 520, FIG. 11 illustrates a mechanical fastener 500 in the form of a snap fastener 530, and FIG. 12 illustrates a mechanical fastener 500 in the form of a synthetic setae fastener 540.

FIG. 9 illustrates a mechanical fastener 500 in the form of a hook-loop fastener 510. A first surface 511 (e.g., defined by one of the base portions 110 or the adhesive patch) has a plurality of hooks 512 projecting therefrom, and a second surface 513 (e.g., defined by the other of the base portion 110 or the adhesive patch) has a plurality of loops 514 projecting therefrom. When the surfaces 511, 513 are pressed together, the hooks 512 engage the loops 514 to releasably secure the surfaces 511, 513 to one another.

FIG. 10 illustrates a mechanical fastener 500 in the form of a hook-hook fastener 520. A first object 521 (e.g., one of the base portion 110 or the adhesive patch) has a first plurality of hooks 522 projecting therefrom, and a second object 523 (e.g., the other of the base portion 110 or the adhesive patch) has a second plurality of hooks 524 projecting therefrom. When the objects 521, 523 are pressed together, the first hooks 522 engage the second hooks 524 to releasably secure the objects 521, 523 to one another.

FIG. 11 illustrates a mechanical fastener 500 in the form of a snap fastener 530. A first object 531 (e.g., one of the base portion 110 or the adhesive patch) has a female snap member 532 mounted thereon, and a second object 533 (e.g., the other of the base portion 110 or the adhesive patch) has a male snap member 534 mounted thereon. When the objects 531, 533 are pressed together, the snap members 532, 534 releasably engage one another to releasably secure the objects 531, 533 to one another.

FIG. 12 illustrates a mechanical fastener 500 in the form of a synthetic setae fastener 540. A first object 541 (e.g., one of the base portion 110 or the adhesive patch) has a plurality of synthetic setae 542 mounted thereon, and a second object 543 (e.g., the other of the base portion 110 or the adhesive patch) comprises an engagement surface 544. The synthetic setae 542 may, for example, be biomimetic structures that mimic the structure and performance of gecko feet. When the objects 541, 543 are pressed together, the synthetic setae 542 engage the engagement surface 544 to releasably secure the objects 541, 543 to one another.

In certain existing approaches to adhering an infusion device to patient skin, the patch is applied after insertion of the needle into patient skin. Certain embodiments of the present application involve attaching the infusion device 100 to patient skin prior to needle insertion, which may facilitate the insertion of the needle at a predetermined angle.

In certain embodiments, the skin-facing adhesive layers may be replaced with mechanical fasteners, such as an array of microneedles. In certain embodiments, the skin-facing adhesive layers may have a therapeutic substance provided therein such that the therapeutic substance begins to release into patient skin upon application of the patch. For example, the therapeutic substance may be a topical anesthetic, sedative, or anti-inflammation agent.

Certain embodiments of the present application relate to an apparatus 80, 80', comprising: a medical device 100 configured to be placed on a patient, the medical device comprising a base portion comprising a patient-facing side configured to face toward patient skin during use of the medical device 100; and an adhesive patch 200, 300, 310 configured to adhere the base portion 110 to patient skin, the adhesive patch 200, 300, 310 comprising a first section 206, 303, 313 and a second section 206', 304, 314; wherein the first section 206, 303, 313 and the second section 206', 304, 314 are separable from one another such that the first section 206, 303, 313 is operable to be removed for replacement while the second section 206', 304, 314 maintains the medical device 100 in a particular position relative to the patient skin.

In certain embodiments, the first section 313 at least partially surrounds the second section 314.

In certain embodiments, the adhesive patch 310 comprises a skin-facing side and 311 a device-facing side 312; wherein the skin-facing side 311 of the first section 313 comprises a first adhesive composition 315'; and wherein the skin-facing side 311 of the second section comprises a second adhesive composition 316' different from the first adhesive composition 315'.

In certain embodiments, the first adhesive composition 315' has a greater adhesion strength than the second adhesive composition 316'.

In certain embodiments, the base portion 110 further comprises a second side 112 opposite the patient-facing side 111; wherein the medical device further comprises a housing 120 positioned on the second side 112 of the base portion 111; and wherein the adhesive patch 200 at least partially surrounds the housing 120.

In certain embodiments, the adhesive patch 200, 300, 310 further comprises a perforation 207, 307, 317 to facilitate separation of the first section 206, 303, 313 and the second section 206', 304, 314. Certain embodiments of the present application relate to an adhesive patch configured adhere a medical device 100 to patient skin, the adhesive patch 400 comprising: a substrate 410 having a first side 412 and a second side 414 opposite the first side 412; a first adhesive layer 420 positioned on the first side 412 of the substrate 410, the first adhesive layer 420 comprising a first adhesive composition 422 configured to adhere to patient skin; and a color-changing composition 440 configured to change colors when exposed to a particular fluid.

In certain embodiments, the adhesive patch further 400 comprises an attachment device 90 configured to attach the substrate 410 to the medical device 100.

In certain embodiments, the attachment device 90 comprises a second adhesive composition 552 different from the first adhesive composition 422.

In certain embodiments, the attachment device 90 comprises at least a portion of a mechanical fastener 500, the at least a portion of the mechanical fastener 500 comprising at least one of: a plurality of hooks 512, 522, 524; a plurality of loops 514; a snap mechanism 532, 534; or synthetic setae 542.

In certain embodiments, the particular fluid is atmospheric air; and wherein the color- changing composition 440 is configured to change colors when exposed to atmospheric air for a predetermined time period.

In certain embodiments, the predetermined time period is three days or longer.

In certain embodiments, the particular fluid is a particular therapeutic agent.

Certain embodiments of the present application relate to an apparatus 80" including the adhesive patch 400, wherein the apparatus 80" further comprises the medical device 100; and wherein the medical device 100 is configured to dispense the particular therapeutic agent.

Certain embodiments of the present application relate to an apparatus 80', comprising: a medical device 100 comprising a base portion 110 having a base portion first side 111 and a base portion second side 112 opposite the base portion first side 111; an adhesive patch 300, 310 configured to be placed between the base portion first side 111 and patient skin, wherein the adhesive patch 300, 310 comprises an adhesive patch first side 301, 311 configured to face patient skin and an adhesive patch second side 302, 312 facing the base portion first side , wherein the adhesive patch first side 301, 311 comprises an adhesive layer 305, 315 comprising a first adhesive composition 305', 315'; and an attachment device 90 attaching the base portion first side 111 to the adhesive patch second side 302, 312, wherein the attachment device 90 comprises at least one of (a) a mechanical fastener 500, or (b) a second adhesive composition different from the first adhesive composition 305', 315'.

In certain embodiments, the attachment device 90 comprises the mechanical fastener 500.

In certain embodiments, the mechanical fastener 500 comprises at least one of a hook- loop fastener 520, a hook-hook fastener 520, a snap 530, or synthetic setae 542.

In certain embodiments, the attachment device 90 comprises the second adhesive composition different from the first adhesive composition 305', 315'.

Certain embodiments of the present application relate to an apparatus 80, 80', comprising: a medical device 100 configured to be placed on a patient, the medical device 100 comprising a base portion 110 comprising a patient-facing side 111 configured to face toward the patient during use of the medical device; and an adhesive patch 200, 300, 310 configured to adhere the base portion 110 to patient skin, the adhesive patch 200, 300, 310 comprising a first region 203, 303, 313 configured for contacting patient skin and a second region 204, 302, 312 attached to the base portion 110 via an attachment device 90, wherein the first region 203, 303, 313 comprises a first adhesive composition 205', 305', 315'; wherein the attachment device 90 comprises at least one of (a) a mechanical fastener 500, or (b) a second adhesive composition different from the first adhesive composition 205', 305', 315'.

In certain embodiments, the attachment device 90 comprises the mechanical fastener 500.

In certain embodiments, the mechanical fastener 500 comprises synthetic setae 542.

In certain embodiments, the mechanical fastener 500 comprises at least one of a hook- loop fastener 510, a hook-hook fastener 520, or a snap 530.

In certain embodiments, the attachment device 90 comprises the second adhesive composition different from the first adhesive composition 205', 305', 315'.

In certain embodiments, the adhesive patch 200 further comprises a first side 201 and an opposite second side 202; and wherein each of the first region 203 and the second region 204 is positioned on the first side 201 of the adhesive patch 200.

In certain embodiments, the first region 203 at least partially surrounds the second region 204.

In certain embodiments, the adhesive patch 300, 310 comprises a first side 301, 311 and an opposite second side 302, 312; wherein the first side 301, 311 of the adhesive patch 300, 310 comprises the first region 305, 315; and wherein the second side 302, 312 of the adhesive patch 300, 310 comprises the second region 302, 312.

In certain embodiments, the adhesive patch 300, 310 further comprises a third region 304, 314 positioned on the first side 301, 311 of the adhesive patch 300, 310; and wherein the third region 303, 313 comprises a third region adhesive composition 306', 316' different from the first adhesive composition 205', 305', 315'.

In certain embodiments, the third region 314 is at least partially surrounded by the first region 313; and wherein the third region adhesive composition 316' has a lesser adhesion strength than the first adhesive composition 315'.

In certain embodiments, the first region 313 has a greater rigidity than the third region 314.

In certain embodiments, the second region 204 has a greater rigidity than the first region 203.

While the invention has been illustrated and described in detail in the drawings and foregoing description, the same is to be considered as illustrative and not restrictive in character, it being understood that only the preferred embodiments have been shown and described and that all changes and modifications that come within the spirit of the inventions are desired to be protected.

It should be understood that while the use of words such as preferable, preferably, preferred or more preferred utilized in the description above indicate that the feature so described may be more desirable, it nonetheless may not be necessary and embodiments lacking the same may be contemplated as within the scope of the invention, the scope being defined by the claims that follow. In reading the claims, it is intended that when words such as "a," "an," "at least one," or "at least one portion" are used there is no intention to limit the claim to only one item unless specifically stated to the contrary in the claim. When the language "at least a portion" and/or "a portion" is used the item can include a portion and/or the entire item unless specifically stated to the contrary.

What is claimed is:

1. An apparatus, comprising:
a medical device configured to be placed on a patient, the medical device comprising a base portion comprising a patient-facing side configured to face toward patient skin during use of the medical device and a second side opposite the patient-facing side; and
an adhesive patch configured to adhere the base portion to the patient skin, the adhesive patch comprising a first section and a second section;
wherein the first section and the second section are separable from one another such that the first section is operable to be removed for replacement while the second section maintains the medical device in a particular position relative to the patient skin, and
wherein the first section and the second section are joined at a perforation that bisects a first region configured to configured to adhere to the patient skin and a second region configured to adhere to the second side of the base portion.

2. The apparatus of claim 1, wherein the first region at least partially surrounds the second region.

3. The apparatus of claim 2, wherein a skin-facing side of the first region comprises a first adhesive composition; and
wherein the second region comprises a second adhesive composition different from the first adhesive composition.

4. The apparatus of claim 3, wherein the first adhesive composition has a greater adhesion strength than the second adhesive composition.

5. The apparatus of claim 1, wherein the medical device further comprises a housing positioned on the second side of the base portion; and
wherein the adhesive patch at least partially surrounds the housing.

6. The apparatus of claim 1, wherein the perforation is configured to facilitate separation of the first section and the second section.

7. An apparatus, comprising:
a medical device comprising a base portion having a base portion first side and a base portion second side opposite the base portion first side;
an adhesive patch configured to be placed between the base portion first side and patient skin, wherein the adhesive patch comprises an adhesive patch first side configured to face the patient skin and an adhesive patch second side facing the base portion first side, wherein the adhesive patch first side comprises an adhesive layer comprising a first adhesive composition disposed on a first section and a second adhesive layer disposed on a second section; and
an attachment device attaching the base portion first side to the adhesive patch second side, wherein the attachment device comprises a mechanical fastener;
wherein the first adhesive composition comprises a greater adhesive strength than the second adhesive composition to discourage edge peeling from the patient skin
wherein perforations are configured to facilitate separation of the first section and the second section, wherein the first section at least partially surrounds the second section.

8. The apparatus of claim 7, wherein the mechanical fastener comprises synthetic setae.

9. The apparatus of claim 1, wherein the medical device further comprises a housing positioned on the second side of the base portion, the housing comprising a reservoir of therapeutic agent and a needle operable to subcutaneously transmit the therapeutic agent to the patient.

10. The apparatus of claim 1, wherein the medical device holds a quantity of therapeutic agent sufficient to last for a predetermined time period of at least three days and wherein the medical device requires reloading or replacement at the end of the predetermined time period.

11. The apparatus of claim 1, wherein the first adhesive composition further comprises an outer periphery comprising a greater adhesive strength than a remainder of the adhesive layer.

12. The apparatus of claim 7, wherein the adhesive patch further comprises a substrate disposed between the adhesive patch first side and the adhesive patch second side, wherein the substrate in the first section comprises a first material of a first, looser weave pattern to provide flexibility and/or breathability to the patient skin, and wherein the substrate in the second section comprises a material formed of a more rigid material of a tighter weave pattern to discourage peeling.

13. The apparatus of claim 7, wherein the attachment device is configured to be positioned at least partially in the first section such that it extends about a periphery of the first section to discourage peeling from the base portion.

14. The apparatus of claim 13, wherein the attachment device is configured to be positioned at least partially in the second section to enhance attachment with the base portion.

15. The apparatus of claim 7, wherein the mechanical fastener comprises a hook and loop fastener.

16. The apparatus of claim 7, wherein the first adhesive composition comprises a non-silicone adhesive and the second adhesive composition comprises a silicon-like adhesive.
